(19) United States
(12) Plant Patent Application Publication
(10) Pub. No.: US 2019/0090396 P1
(43) Pub. Date: Mar. 21, 2019

(54) Miniature rose plant named 'Meibenbino'

(50) ;

(71) Applicant: THE CONARD-PYLE COMPANY, West Grove, PA (US)

(72) Inventor: Alain Antoine Meilland, Antibes (FR)

(73) Assignee: THE CONARD-PYLE COMPANY, West Grove, PA (US)

(21) Appl. No.: 15/932,925

(22) Filed: May 23, 2018

(30) Foreign Application Priority Data
Sep. 21, 2017 (EP) 2017-2341

(51) Int. Cl. A01H 6/74

(52) U.S. Cl. PLT/122

(57) ABSTRACT

A new and distinct variety of Miniature Rose plant, herein referred to by its cultivar name, ‘MEIBENBINO’, is provided which forms very abundantly on a nearly continuous basis attractive, cup-shaped, red colored blossoms. Attractive, glossy, very dense foliage is formed, which contrasts beautifully with the blossoms. The disease tolerance is very good, particularly against Black Spot. The new variety is well suited for proving attractive ornamentation in the landscape.

### BOTANICAL/COMMERCIAL CLASSIFICATION

Latin name—Rosa hybrida.

Common name—Miniature Rose Plant.

Varietal denomination—‘MEIBENBINO’.

### CROSS-REFERENCE TO RELATED APPLICATIONS

This application claims priority to Community Plant Variety Rights application number 2017/2341 filed on Sep. 21, 2017, which is incorporated herein by reference in its entirety for all purposes.

### SUMMARY OF THE INVENTION

The new variety of Rosa hybrida Miniature Rose plant was created by artificial pollination wherein two parents were crossed which previously had been studied in the hope that they would contribute the desired characteristics. The female parent (i.e., the seed parent) was the product of a cross of the ‘MEIPAONIA’ variety (non-patented in the United States) and an ‘UNNAMED SEEDLING’ variety. The male parent (i.e., the pollen parent) was the ‘RADTKO’ variety (U.S. Plant Pat. No. 16,202).

The parentage of the new variety can be summarized as follows:

(‘MEIPAONIA’ x ‘UNNAMED SEEDLING’) x ‘RADTKO’

The seeds resulting from the above pollination were sown and small plants were obtained which were physically and biologically different from each other. Selective study resulted in the identification of a single plant of the new variety.

It was found that the new variety of Miniature Rose plant of the present invention possesses the following combination of characteristics:

(a) very abundantly and nearly continuously forms attractive, cup-shaped, red colored blossoms,

(b) forms attractive, very dense, glossy foliage,

(c) exhibits very good tolerance to diseases, particularly against Black Spot, and

(d) is well suited for providing attractive ornamentation.

The new variety well meets the needs of the horticultural industry. It can be grown to advantage as ornamentation in parks, gardens, public areas, and in residential settings. Accordingly, the plant is particularly well suited for growing in the landscape.

The new variety of the present invention can readily be distinguished from its ancestors. More specifically, the ‘RADTKO’ variety (i.e., the pollen parent) is a shrub rose plant/bedding rose plant, whereas the new variety is a miniature rose plant. The ‘MEIPAONIA’ variety (i.e., a parent of the seed parent) exhibits ocher colored blossoms, whereas the new variety exhibits red colored blossoms.

The new variety has been found to undergo asexual propagation in France by a number of routes, including budding, grafting, and the use of cuttings. Asexual propagation by the above-mentioned techniques in France has shown that the characteristics of the new variety are stable and are strictly transmissible by such asexual propagation from one generation to another. Accordingly, the new variety undergoes asexual propagation in a true-to-type manner.

The new variety has been named ‘MEIBENBINO’.

### BRIEF DESCRIPTION OF THE PHOTOGRAPH

The accompanying photographs show as nearly true as it is reasonably possible to make the same, in a color illustration of this character, typical specimens of the plant parts of the new variety. The rose plants of the new variety were approximately one year of age and were observed during July while budded on their own roots and growing outdoors at Le Cannet des Maures, Var, France. Standard color information and dimensions in centimeters are included at the bottom of the photograph.

FIG. 1—illustrates a specimen of a young shoot.

FIG. 2—illustrates a specimen of a floral bud before the opening of the sepals.

FIG. 3—illustrates a specimen of a floral bud at the opening of the sepals.

FIG. 4—illustrates a specimen of a floral bud at the opening of the petals.

FIG. 5—illustrates a specimen of a flower in the course of opening.

FIG. 6—illustrates a specimen of a flower in the course of opening—plan view—obverse.

FIG. 7—illustrates a specimen of a flower in the course of opening—plan view—reverse.

FIG. 8—illustrates a specimen of a fully open flower—plan view—obverse.

FIG. 9—illustrates a specimen of a fully open flower—plan view—reverse.

FIG. 10—illustrates a specimen of a floral receptacle showing arrangement of the stamens and pistils.

FIG. 11—illustrates a specimen of a floral receptacle showing arrangement of the pistils (stamens removed).

FIG. 12—illustrates a specimen of a flowering stem.

FIG. 13—illustrates a specimen of a leaf with 3 leaflets—plan view—upper surface.

FIG. 14—illustrates a specimen of a leaf with 5 leaflets—plan view—under surface.

FIG. 15—illustrates a specimen of a leaf with 7 leaflets—plan view—upper surface.

FIG. 16—illustrates a specimen of buds in cluster.

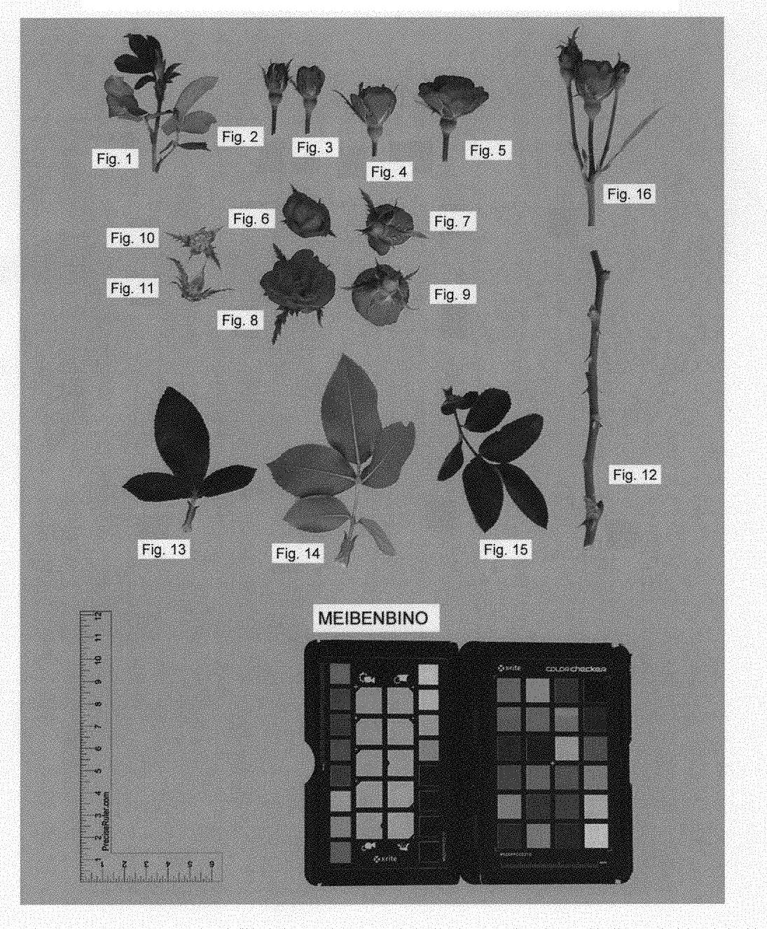

### DETAILED BOTANICAL DESCRIPTION

The chart used in the identification of the colors is that of The Royal Horticultural Society (R.H.S. Color Chart), London, England. The terminology which precedes reference to the chart has been added to indicate the corresponding color in more common terms. The description is based on the observation of one-year-old specimens of the new variety during July while budded on their own roots and growing outdoors at Le Cannet des Maures, Var, France.
Class: Miniature Rose Plant.
Plant:
   Habit.—Semi-erect.
Branches:
   Color.—Young stems: commonly near Yellow-Green Group 147C. — adult wood commonly near Yellow-Green Group 146C.
   Thorns.—Configuration on adult stems: rather upright on the upper surface and slightly concave on the under surface. — long prickles (≥0.6 cm) — quantity: commonly approximately 5 thorns per 10 cm long young stem and approximately 6 thorns per 10 cm long adult stem. — long prickles (≥0.6 cm) — base shape: oval. — long prickles (≥0.6 cm) — size: commonly near 0.6 cm in length on average on young stems and commonly near 0.7 cm in length on average on adult stems. — long prickles (≥0.6 cm) — color: commonly near Yellow-Green Group 147D on young stems and commonly near Greyed-Orange Group 164A on adult stems. — small prickles (<0.6 cm) — none observed.
Foliage:
   General appearance.—Very dense with a glossy aspect.
   Number of leaflets.—3, 5, and 7; most often 5 or 7.
   Terminal leaflet.—length: approximately 3.7 cm on average. — width: approximately 2.3 cm on average.
   New foliage.—Upper surface color: commonly near Yellow-Green Group 147B. — under surface color: commonly near Yellow-Green Group 146C.
   Adult foliage.—Upper surface color: commonly near Green Group 137A. — under surface color: commonly near Yellow-Green Group 146B.
Leaflets:
   Shape tip.—Acuminate. — base: cuneiform.
   Texture.—Leathery.
   General appearance.—Elliptical.
   Edge.—Slightly denticulate.
   Serration.—Small and single.
   Petiole rachis.—Color of upper surface: commonly near Greyed-Orange Group 164A. — color of under surface: commonly near Yellow-Green Group 146B.
   Petioles.—Upper surface: no glandular. — under surface: few prickles. — color of upper surface: commonly near Yellow-Green Group 146B. — color of under surface: commonly near Yellow-Green Group 146B. — petiole length of terminal leaflet: commonly near 1.9 cm on average.
   Stipules.—General appearance: adnate, pectinate and narrow. — length: approximately 0.7 cm on average. — width: approximately 0.3 cm on average. — color of upper surface: commonly near Yellow-Green Group 146B. — color of under surface: commonly near Yellow-Green Group 146B.
Inflorescence:
   Number of flowers per stem.—Generally between 1 and 5 flowers per stem.
   Bud.—Shape: conical. — size: small. — length: approximately 1.0 cm on average. — width: approximately 0.9 cm on average. — color as calyx breaks: upper surface: commonly near Red Group 46B to Red Group 46A. under surface: commonly near Red Group 46B to Red Group 46A.
   Sepals.—Length: approximately between 1.3 cm on average. — width: approximately near 0.4 cm on average. — shape: some longish and narrow denticulate extensions, at the top: longish and narrow, at the base: upright. — upper surface: texture: tomentous. color: commonly near Yellow-Green Group 148C. — under surface: texture: smooth. color: commonly near Yellow-Green Group 146A.
   Receptacle.—Color: commonly near Yellow-Green Group 146A. — length: approximately 0.6 cm on average. — width: approximately 0.7 cm on average. — surface: smooth. — shape: funnel shaped.
   Peduncle.—Length: approximately 2.7 to 3.3 cm on average. — width: approximately 0.3 cm on average. — surface: smooth. — color: commonly near Yellow-Green Group 146A.
   Flower.—Average diameter when fully open: approximately 3.0 to 3.5 cm on average. — borne: commonly between 1 to 5 flowers per stem. — shape: cup shaped. — number of petals under normal conditions: approximately 9 to 12 petals on average. — shape of the petal: base: obtuse. top: rounded. — texture of the petal: leathery. — petal length: approximately 2.4 cm on average. — petal width: approximately 2.2 cm on average. — petal arrangement: imbricated with some petaloids. — petal drop: petals drop off cleanly before drying. — fragrance: none. — color when opening: upper surface: commonly near Red Group 46B to Red Group 46A, very little spot at the base commonly near Yellow Group 13D. under surface: commonly near Red Group 46B to Red Group 46A, very little spot at the base commonly near Yellow Group 13D. — color of the open flower: upper surface: commonly near Red Group 46B to Red Group 46A, very little spot at the base commonly near Yellow Group 13D. under surface: commonly near Red Group 46B to Red Group 46A, very little spot at the base commonly near Yellow Group 13D. — anthers: number is approximately 52 on average, length is approximately 0.1 cm on average, coloration is commonly near Greyed-Orange Group 173B, and arrangement is regular around styles. — filaments: length is approximately near 0.3 cm on average and coloration is commonly near Red Group 42D. — styles: length is approximately 0.4 cm on average, the coloration is commonly near Red Group 44C, and the number is approximately 32 on average. — stigmas: size is approximately 0.1 cm on average and the coloration is commonly near Yellow Group 12D. — pollen: not available at this stage. — hips: not available at this stage.
Development:
   Vegetation.—Strong.
   Blooming.—Early in the season, very abundant and nearly continuous.
   Tolerance to disease.—Very good, and particularly against Black Spot.

The new ‘MEIBENBINO’ variety has not been observed under all possible environmental conditions to date. Accordingly, it is possible that the phenotypic expression may vary somewhat with changes in light intensity and duration, cultural practices, and other environmental conditions.

I claim:

1. A new and distinct variety of Miniature Rose plant characterized by the following combination of characteristics:
(a) very abundantly and nearly continuously forms attractive, cup-shaped, red colored blossoms,
(b) forms attractive, very dense, glossy foliage,
(c) exhibits very good tolerance to diseases, particularly against Black Spot, and
(d) is well suited for providing attractive ornamentation;
substantially as herein shown and described.
